(19) United States
(12) Patent Application Publication
(10) Pub. No.: US 2023/0293511 A1
(43) Pub. Date: Sep. 21, 2023

(54) Ophthalmic Compositions and Uses Thereof

(71) Applicant: Richard L. Lindstrom, Wayzata, MN (US)

(72) Inventor: Richard L. Lindstrom, Wayzata, MN (US)

(21) Appl. No.: 18/122,417

(22) Filed: Mar. 16, 2023

Related U.S. Application Data
(60) Provisional application No. 63/321,287, filed on Mar. 18, 2022.

(51) Int. Cl. A61K 31/47; A61K 47/10; A61K 47/36; A61K 47/26; A61K 9/00; A61P 27/02

(52) U.S. Cl. CPC A61K 31/47; A61K 47/10; A61K 47/36; A61K 47/26; A61K 9/0048; A61P 27/02

(57) ABSTRACT

Ophthalmic compositions including an RKI, a lubricant, a deturgescent agent, a glycosaminoglycan, and water. Methods of using the ophthalmic compositions are also provided.

### CROSS REFERENCE TO RELATED APPLICATION

This application claims the benefit of, and priority to, U.S. Provisional Patent Application Ser. No. 63/321,287, filed on Mar. 18, 2022, the entire disclosure of which is hereby incorporated by reference in its entirety.

### FIELD

Provided herein are ophthalmic compositions that include a rho kinase inhibitor (RKI), a cell culture media, a deturgescent agent, a glycosaminoglycan, and a liquid carrier. Methods of making and using the ophthalmic compositions are also provided.

### BACKGROUND

Ophthalmic compositions are used to treat a variety of conditions affecting the eye, ranging from “dry eye” to glaucoma. However, there remains a need for effective compositions and methods to preserve ocular tissues prior to transplant, for example to condition a cornea (the transparent “front” part of the eye that covers the iris, pupil, and anterior chamber) prior to surgery, as well as protect and rehabilitate the ocular tissue following the trauma associated with a surgical transplant procedure.

It is also important to enhance recovery during the healing period following ocular tissue transplant surgery, as well as to improve vision by reducing tissue edema, regenerating cell numbers, health, and function, stabilizing cell membranes, and scavenging free radicals in the traumatized surface.

### SUMMARY

Disclosed herein are ophthalmic compositions comprising a rho kinase inhibitor (RKI), a cell culture media, a deturgescent agent, a glycosaminoglycan, and a liquid carrier.

For example, disclosed compositions can comprise;
a. an RKI;
b. a cell culture media such as Minimum Essential Medium (MEM);
c. a deturgescent agent such as dextran;
d. a glycosaminoglycan such as chondroitin sulfate; and
e. a liquid carrier such as water.

Further disclosed compositions can comprise;
a. An RKI;
b. a buffered corneal preservation medium;
c. recombinant human insulin (cell metabolism enhancer);
d. glutathione (antioxidant, free-radical scavenger, enzyme cofactor);
e. stabilized L-glutamine;
f. ATP precursors;
g. nutrient cell supplements;
h. amino acids;
i. vitamins;
j. trace elements;
k. gentamicin;
l. streptomycin; and
m. phenol red (pH indicator).

Further disclosed compositions can comprise;
a. an RKI;
b. a cell culture media such as Minimum Essential Medium (MEM);
c. a deturgescent agent such as dextran;
d. a glycosaminoglycan such as chondroitin sulfate;
e. a liquid carrier such as water;
f. a buffered corneal preservation medium;
g. recombinant human insulin (cell metabolism enhancer);
h. glutathione (antioxidant, free-radical scavenger, enzyme cofactor);
i. stabilized L-glutamine;
j. ATP precursors;
k. nutrient cell supplements;
l. amino acids;
m. vitamins;
n. trace elements;
o. gentamicin;
p. streptomycin; and
q. phenol red (pH indicator).

Disclosed compositions can comprise an RKI in an amount from 1 to 100 mM, dextran in a concentration of from 0.1% to 10.0%, chondroitin sulfate in a concentration of from 0.1% to 5.0%, and water.

Further disclosed are methods of treating the cornea. For example, disclosed embodiments comprise methods of treating corneal edema. In embodiments, the method comprises administering to a cornea during storage and transportation prior to surgery of a subject suffering from or susceptible to corneal edema an effective amount of an ophthalmic composition comprising an RKI in an amount from 1 to 100 mM, dextran in a concentration of from 1.0% to 10.0%, chondroitin sulfate in a concentration of from 0.1% to 5.0%, and water.

Further disclosed are methods of conditioning a donor cornea prior to transplantation. In embodiments, the method comprises administering to a donor cornea an effective amount of an ophthalmic composition comprising an RKI in an amount from 1 to 100 mM, dextran in a concentration of from 1.0% to 10.0%, chondroitin sulfate in a concentration of from 0.1% to 5.0%, and water.

Further disclosed are methods of making the disclosed compositions.

Further disclosed are methods of improving the performance of a current ophthalmic solution by adding to the solution 1 mM to 100 mM of an RKI.

Further disclosed are kits comprising the disclosed compositions as well as instructions for use.

### BRIEF DESCRIPTION OF THE DRAWINGS

The following drawings form part of the present specification and are included to further demonstrate certain aspects of the examples disclosed herein. The invention may be better understood by reference to one or more of these drawings in combination with the detailed description of specific embodiments presented herein.

FIG. 1 is a graphic representation of the study described in Example 6.

FIG. 2 shows netarsudil QD (once a day dosage) and BID (twice a day dosage) significantly improved CCT from baseline at weeks 2, 4, and 8, without significant differences between QD and BID dosing.

FIG. 3 shows netarsudil QD improved BCVA (best corrected visual acuity) by 5.0 and 3.2 letters at weeks 2 and 4, respectively.

FIG. 4; overall, 25% of patients gained letters in BCVA at week 4.

FIG. 5; improvements in patient-reported outcomes with V-FUCHS Questionnaire were shown at all timepoints with netarsudil QD.

### DETAILED DESCRIPTION

The present disclosure relates to ophthalmic compositions as well as methods of making and using the same.

Disclosed Compositions

Disclosed compositions improve upon currently available corneal preservation media such as OPTISOL® GS, KLARITY®, or LIFE4C. For example, disclosed compositions enhance maintenance of endothelial cell viability during corneal preservation and transportation at 4° to 40° C.

Further, in embodiments the RKI enhances early post-transplant surgery healing of the critical endothelial cell layer by enhancing endothelial cell adhesion, migration and cell division. Thus, disclosed compositions and methods can produce a donor cornea with a higher endothelial cell count after surgery, as well as more rapid healing and faster visual recovery.

Further embodiments comprise topical lubricants, drug formulation vehicles, ocular irrigating solutions, and viscoelastic materials.

Further embodiments comprise the augmentation of current products such as OPTISOL® GS, LIFE4C, and KLARITY®. For example, in embodiments, a current ophthalmic solution is augmented with an RKI, for example in a concentration of between 1 mM and 100 mM. RKI augmentation can increase endothelial and epithelial cell viability and wound healing during corneal storage, after surgical trauma or after any other disease related injury including corneal dystrophy, Dry Eye Disease, or Inflammation (Uveitis).

Disclosed herein are ophthalmic compositions consisting of, consisting essentially of, or comprising one or more of the following:
a. a rho kinase inhibitor (RKI);
b. a cell culture medium;
c. a deturgescent agent;
d. a glycosaminoglycan; and
e. a liquid carrier.

Disclosed herein are ophthalmic compositions consisting of, consisting essentially of, or comprising one or more of the following:
a. An RKI;
b. a buffered corneal preservation medium;
c. recombinant human insulin (cell metabolism enhancer);
d. glutathione (antioxidant, free-radical scavenger, enzyme cofactor);
e. stabilized L-glutamine;
f. ATP precursors;
g. nutrient cell supplements;
h. amino acids;
i. vitamins;
j. trace elements;
k. gentamicin;
l. streptomycin; and
m. phenol red (pH indicator).

Disclosed herein are ophthalmic compositions consisting of, consisting essentially of, or comprising one or more of the following:
a. an RKI;
b. a cell culture media such as Minimum Essential Medium (MEM);
c. a deturgescent agent such as dextran;
d. a glycosaminoglycan such as chondroitin sulfate;
e. a liquid carrier such as water;
f. a buffered corneal preservation medium;
g. recombinant human insulin (cell metabolism enhancer);
h. glutathione (antioxidant, free-radical scavenger, enzyme cofactor);
i. stabilized L-glutamine;
j. ATP precursors;
k. nutrient cell supplements;
l. amino acids;
m. vitamins;
n. trace elements;
o. gentamicin;
p. streptomycin; and
q. phenol red (pH indicator).

Disclosed embodiments comprise an RKI, for example, one of the following:
a. AT-13148;
b. BA-210;
c. β-Elemene;
d. Chroman 1;
e. DJ4;
f. Fasudil;
g. GSK-576371;
h. GSK429286A, C₂₁H₁₆F₄N₄O₂;
i. H-1152;
j. Hydroxyfasudil;
k. Ibuprofen;
l. LX-7101;
m. Netarsudil;
n. RKI-1447;
o. Ripasudil;
p. TCS-7001;
q. Thiazovivin;
r. Verosudil (AR-12286);
s. Y-27632;
t. Y-30141;
u. Y-33075;
v. Y-39983.

Rho kinases are serine/threonine kinases involved in regulating the shape and movement of cells, mainly by acting on the cytoskeleton. RKIs comprise a series of compounds that target rho kinases, and alter the conformation of the enzymes, thus interrupting their action. While the use of RKIs has been reported, their ophthalmic use has been associated with conjunctival hyperemia (ocular redness), present in more than half of the patients using certain formulations. In contrast, disclosed compositions comprise additional components that mitigate the risk of conjunctival hyperemia while providing increased effectiveness as compared to known formulations.

In embodiments, disclosed compositions comprise a lubricant, for example at least one of glycerol, hydroxypropylmethyl cellulose, carboxy propylmethyl cellulose, sorbitol, polyvinyl pyrrolidone, polyethylene glycol, polyvinyl acetate, and combinations thereof.

In embodiments, disclosed compositions comprise a deturgescent agent, for example at least one of dextran, dextran sulfate, NaCl, dextrose, sucrose, other sugars, and combinations thereof. In embodiments, any suitable molecular weight dextran or mixture thereof may be used, including for example dextran 40, dextran 70, and/or dextran 500.

In embodiments, disclose compositions comprise at least one glycosaminoglycan such as chondroitin sulfate, chondroitin, dermatan sulfate, dermatin sulfate, heparin sulfate, heparan sulfate, keratin sulfate, keratan sulfate, hyaluronic acid, and mixtures thereof. In embodiments, any one isomer or salt of the glycosaminoglycan may be used, or a mixture of isomers and/or salts of the glycosaminoglycan may be used. For example, as used herein, “chondroitin sulfate” comprises any type of chondroitin sulfate, including isomers and salts thereof as well as mixtures of isomers and/or salts thereof.

In embodiments, disclosed compositions comprise a liquid carrier. For example, in embodiments the liquid carrier comprises water, for example sterilized water.

Thus, in embodiments, disclosed compositions can comprise an RKI, glycerol, dextran, chondroitin sulfate, and water. For example, disclosed embodiments can comprise an RKI in an amount of from 1 to 100 mM, such as 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, or more.

In embodiments, the amount of the RKI can be between 1 mM and 20 mM, between 3 mM and 17 mM, between 6 mM and 14 mM, between 9 mM and 11 mM, or the like.

In embodiments, the amount of the RKI can be not less than 5 mM, not less than 10 mM, not less than 15 mM, or the like.

In embodiments, disclosed compositions comprise a lubricant. For example, in embodiments, the lubricant can comprise glycerol in an amount of, for example, 0.05%, 0.1%, 0.2%. 0.3%, 0.4%, 0.5%, 0.6%, 0.7%. 0.8%, 0.9%, 1%, 2%, 3%, 4%, 5%, 6%, 7%, 8%, 9%, 10%, or the like.

In embodiments, the amount of lubricant in the composition can be, for example, between 0.05% and 1.5%.

In embodiments, the amount of the lubricant can be, for example, not less than 0.05%, not less than 0.1%, not less than 0.5%, not less than 1%, or the like.

Further embodiments comprise a deturgescent agent, for example dextran. In embodiments, the amount of deturgescent agent can be 0.025%, 0.05%, 0.1%, 0.2%. 0.3%, 0.4%, 0.5%, 0.6%, 0.7%. 0.8%, 0.9%, 1%, 2%, 3%, 4%, 5%, 6%, 7%, 8%, 9%, 10%, 11%, 12%, 13%, 14%, 15%, or the like.

In embodiments, the amount of deturgescent agent can be between 0.025% and 15%.

Disclosed compositions can comprise a glycosaminoglycan such as chondroitin sulfate. In embodiments, the amount of glycosaminoglycan can be, for example, 0.05%, 0.1%, 0.2%. 0.3%, 0.4%, 0.5%, 0.6%, 0.7%. 0.8%, 0.9%, 1%, 2%, 3%, 4%, 5%, 6%, 7%, 8%, 9%, 10%, or the like.

In embodiments, the amount of glycosaminoglycan in the composition can be, for example, between 0.05% and 1.5%.

In embodiments, the amount of the glycosaminoglycan can be, for example, not less than 0.05%, not less than 0.1%, not less than 0.5%, not less than 1%, or the like.

Disclosed embodiments can further comprise water, for example sterilized water.

In some embodiments, the ophthalmic composition consists essentially of an RKI, a lubricant, a deturgescent agent, a glycosaminoglycan, and water. The compositions can also comprise a buffer (e.g., buffers including citrates, phosphates, borates, bicarbonates, sodium salts, potassium salts, etc.), an acid or base to modify pH, a tonicity modulating agent (e.g., NaCl), and/or an antioxidant/free radical scavenger (e.g., ascorbate, ascorbic acid, glutathione, etc.). In other embodiments, the ophthalmic compositions consist of an RKI, a lubricant, a deturgescent agent, a glycosaminoglycan, water, a buffer, and a tonicity modulating agent. In yet other embodiments, the ophthalmic compositions consist of an RKI, a lubricant, a deturgescent agent, a glycosaminoglycan, and water.

In embodiments, the ophthalmic composition comprises an RKI, glycerol, dextran, chondroitin sulfate, and water. The RKI is typically present in an amount of from 1 mM to 100 mM, such as 5 mM to 20 mM, or 10 mM. The glycerol is typically present in such a composition in a concentration of from 0.1% to 5.0%, such as from 0.3% to 1.7%, or from 0.6% to 1.4%, or from 0.8% to 1.2%, or 1.0%. Dextran is typically present in such a composition in a concentration of from 0.1% to 10%, such as from 0.5% to 10%, or from 1.0% to 10.0%, or from 3% to 7%, or from 4% to 6%, or from 4.5% to 5.5%, or in a concentration of 5.0%. Chondroitin sulfate is typically present in such a composition in a concentration of from 0.1% to 5.0%, such as from 0.5% to 4.5%, or from 1.5% to 3.5%, or from 2.0% to 3.0%, or from 2.3% to 2.7%, or in a concentration of 2.5%.

Disclosed ophthalmic compositions typically have a pH from 5.0 to 9.0, such as from 6.0 to 8.0, or from 7.0 to 7.4, or 7.0, although the compositions may also have a pH outside of these ranges. In embodiments, a buffer (e.g., a buffer with intrinsic antimicrobial properties such as a sodium borate/boric acid buffer) may be used to achieve (and maintain) the desired pH of the compositions, and/or an acid or base may be added to adjust the pH of the compositions to the desired level. In embodiments, buffers that do not require adjustment of the pH of the compositions with additional acid or base are preferred.

Disclosed ophthalmic compositions typically have an osmolarity of from 100 to 500 milliosmoles/liter (mOsm/L), such as from 150 to 450 mOsm/L, or from 200 to 400 mOsm/L, although the compositions may also have an osmolarity outside of these ranges. A tonicity modulating agent such as sodium chloride may also be used in the compositions.

As stated above, in some embodiments, disclosed ophthalmic compositions comprise an RKI, glycerol, dextran, chondroitin sulfate, and water, and optionally includes a buffer, an acid or base, a tonicity modulating agent, and/or a free radical scavenger. In other embodiments, the ophthalmic composition consists essentially of an RKI, glycerol, dextran, chondroitin sulfate, and water, and optionally includes a buffer, an acid or base, a tonicity modulating agent, and/or a free radical scavenger. In further embodiments, the composition consists of an RKI, glycerol, dextran, chondroitin sulfate, and water, and optionally includes a buffer, an acid or base, a tonicity modulating agent, and/or a free radical scavenger. In yet other embodiments, the composition consists of an RKI, glycerol, dextran, chondroitin sulfate, and water.

Methods of Producing Disclosed Compositions

Any effective method may be used to produce the ophthalmic compositions described herein. For example, any effective method may be used to produce a composition consisting of, consisting essentially of, or comprising:
a. a rho kinase inhibitor (RKI);
b. a lubricant or cell culture medium;
c. a deturgescent agent;
d. a glycosaminoglycan; and
e. a liquid carrier.

Similarly, any effective method may be used to produce a composition consisting of, consisting essentially of, or comprising:
a. An RKI;
b. a buffered corneal preservation medium;
c. recombinant human insulin (cell metabolism enhancer);
d. glutathione (antioxidant, free-radical scavenger, enzyme cofactor);
e. stabilized L-glutamine;
f. ATP precursors;
g. nutrient cell supplements;
h. amino acids;
i. vitamins;
j. trace elements;
k. gentamicin;
l. streptomycin; and
m. phenol red (pH indicator).

Similarly, any effective method may be used to produce a composition consisting of, consisting essentially of, or comprising:
a. an RKI;
b. a cell culture media such as Minimum Essential Medium (MEM);
c. a deturgescent agent such as dextran;
d. a glycosaminoglycan such as chondroitin sulfate;
e. a liquid carrier such as water;
f. a buffered corneal preservation medium;
g. recombinant human insulin (cell metabolism enhancer);
h. glutathione (antioxidant, free-radical scavenger, enzyme cofactor);
i. stabilized L-glutamine;
j. ATP precursors;
k. nutrient cell supplements;
l. amino acids;
m. vitamins;
n. trace elements;
o. gentamicin;
p. streptomycin; and
q. phenol red (pH indicator).

An example of a method for making a disclosed ophthalmic composition is disclosed in Example 1.

Methods of Use of Disclosed Compositions

Disclosed methods comprise use of a composition consisting of, consisting essentially of, or comprising:
a. a rho kinase inhibitor (RKI);
b. a lubricant or cell culture medium;
c. a deturgescent agent;
d. a glycosaminoglycan; and
e. a liquid carrier.

Further disclosed methods comprise use of a composition consisting of, consisting essentially of, or comprising:
a. An RKI;
b. a buffered corneal preservation medium;
c. recombinant human insulin (cell metabolism enhancer);
d. glutathione (antioxidant, free-radical scavenger, enzyme cofactor);
e. stabilized L-glutamine;
f. ATP precursors;
g. nutrient cell supplements;
h. amino acids;
i. vitamins;
j. trace elements;
k. gentamicin;
l. streptomycin; and
m. phenol red (pH indicator).

Further disclosed methods comprise use of a composition consisting of, consisting essentially of, or comprising:
a. an RKI;
b. a cell culture media such as Minimum Essential Medium (MEM);
c. a deturgescent agent such as dextran;
d. a glycosaminoglycan such as chondroitin sulfate;
e. a liquid carrier such as water;
f. a buffered corneal preservation medium;
g. recombinant human insulin (cell metabolism enhancer);
h. glutathione (antioxidant, free-radical scavenger, enzyme cofactor);
i. stabilized L-glutamine;
j. ATP precursors;
k. nutrient cell supplements;
l. amino acids;
m. vitamins;
n. trace elements;
o. gentamicin;
p. streptomycin; and
q. phenol red (pH indicator).

For example, use of disclosed compositions can comprise administration via methods known in the art, such as, for example, topical administration.

Disclosed ophthalmic compositions are useful for preventative and therapeutic treatment of numerous ocular conditions and diseases as well as before, during, and after various ocular surgeries, as the compositions provide for increased corneal endothelial cell recovery and healing including cell number and viability, ocular surface lubrication, cell membrane stabilization, corneal deturgescence, and, when the ophthalmic compositions contain an antioxidant/free radical scavenger, antioxidant activity.

Disclosed ophthalmic compositions are useful for protecting the ocular surface (e.g., cornea and conjunctiva), corneal epithelial cells, corneal endothelial cells, and/or other ocular tissues during surgery on an eye. In addition to preventing damage to such ocular tissues during corneal preservation and surgery, the ophthalmic compositions may be useful in wound healing after surgery or other events causing injury to the eye. The ophthalmic compositions may also be useful for reducing corneal edema (e.g., during and after corneal transplantation surgery) as well as maintaining corneal deturgescence.

Disclosed methods of use of disclosed ophthalmic compositions can comprise methods of protecting an animal (e.g., a mammal, especially a human) ocular surface, cornea, corneal epithelial cells, corneal endothelial cells, and/or other ocular tissue comprise administering an effective amount of an ophthalmic composition described herein to the ocular surface, cornea, corneal epithelial cells, corneal endothelial cells, and/or other ocular tissue of a subject. Such administration may occur before and/or during events such as surgery that may cause trauma to the ocular surface, cornea, corneal epithelial cells, corneal endothelial cells, and/or other ocular tissue. The ophthalmic composition preferably maintains contact with the ocular surface, cornea, corneal epithelial cells, corneal endothelial cells, and/or other ocular tissue during the trauma causing event (e.g., surgery).

The ophthalmic compositions may further be used as a general surgical rinsing solution, especially during ocular surgeries. In addition, the ophthalmic compositions may be useful for rehabilitating stressed or damaged ocular tissue (e.g., an ocular surface) to a normal state (i.e., homeostasis). For example, the ophthalmic compositions may be useful for rehabilitating the ocular surface before and after contact lens wear. Furthermore, the ophthalmic compositions may be useful for maintaining ocular tissue (e.g., an ocular surface) at a normal state (i.e., homeostasis). The ophthalmic compositions may also be useful for enhancing comfort during contact lens wear.

Disclosed methods of use of disclosed ophthalmic compositions can comprise methods of treating wounds and/or promoting healing after events causing trauma to an animal (e.g., a mammal, especially a human) ocular surface, cornea, corneal epithelial cells, corneal endothelial cells, and/or other ocular tissue comprise administering an effective amount of an ophthalmic composition described herein to the ocular surface, cornea, corneal epithelial cells, corneal endothelial cells, and/or other ocular tissue of a subject. Such administration may occur before, during, or after events such as surgery that may cause trauma to such ocular tissues, and the ophthalmic composition preferably maintains contact with such ocular tissues after the trauma causing event (e.g., ocular surgery).

Disclosed methods comprising a corneal transplant can increase the number and viability of corneal endothelial cells, thus improving patient outcomes, for example in terms of the lifespan of the transplant.

Disclosed methods of use of disclosed ophthalmic compositions can comprise methods of reducing corneal edema comprise administering an effective amount of an ophthalmic composition described herein to an animal (e.g., a mammal, especially a human) cornea of a subject suffering from or susceptible to corneal edema in order to reduce and/or prevent edema in the cornea. Such corneal edema may be caused by an event or disease causing corneal edema such as, for example, corneal transplantation surgery or corneal edema occurring spontaneously. The ophthalmic composition may be administered to the cornea before and/or after edema occurs (e.g., before, during, and/or after corneal transplantation surgery) in order to reduce and/or prevent corneal edema resulting from events such as surgery anticipated to cause such edema.

Disclosed methods of use of disclosed ophthalmic compositions can comprise methods of maintaining ocular tissue (e.g., an ocular surface) at a normal state (i.e., homeostasis) comprise administering an effective amount of an ophthalmic composition described herein to the ocular tissue (e.g., ocular surface). Such a method may be used before, during, or after stress or other damage to the ocular tissue (e.g., ocular surface) such as before, during, and after contact lens wear.

Disclosed methods of use of disclosed ophthalmic compositions can comprise methods of enhancing comfort during contact lens wear comprise administering an effective amount of an ophthalmic composition described herein to the ocular surface and/or other ocular tissue of a subject wearing one or more contact lenses.

In embodiments, disclosed ophthalmic solutions may be administered as a single dosage, in periodic applications, or may be maintained on the ophthalmic tissue continuously or substantially continuously as appropriate for the particular use. For example, the ophthalmic compositions may be administered once per day in some embodiments, may be administered once every minute for a period of 5 to 10 minutes in other embodiments, and may be administered more or less frequently in yet other embodiments. For methods of maintaining ocular tissue at a normal state as well as in other embodiments of methods of rehabilitating stressed or damaged ocular tissue, an effective amount of the ophthalmic compositions may be applied between 1 to 16 times a day (e.g., from 1 to 8 times a day, from 1 to 6 times a day, or from 1 to 4 times a day), although the ophthalmic compositions may be administered more or less frequently in methods of maintaining ocular tissue as well as in other methods. As will be understood, an effective amount of ophthalmic composition will vary depending upon the particular use, the particular patient and eye the composition is being applied to, and other variable factors. For example, for methods of rehabilitating stressed or damaged ocular tissue, two or three drops of the ophthalmic composition may be used immediately after an insult and could be administered every minute (or other interval) for a period thereafter, although other amounts of ophthalmic compositions could be used in more or less frequency.

In particular, the ophthalmic compositions of the present invention are useful in ophthalmic surgery because, when placed on the patient's cornea immediately prior to and/or during surgery, the surgeon's view of intraocular structures during surgery is facilitated and enhanced by the presence of the ophthalmic solution of the present invention upon the cornea, thereby rendering the surgeon more effective and efficient. Additionally, the presence of the ophthalmic solution of the present invention upon the cornea protects the corneal epithelium and conjunctival epithelium and reduces intraoperative damage to the corneal epithelial and conjunctival epithelial cells. Moreover, the presence of the ophthalmic solution of the present invention upon the cornea reduces intraoperative development of epithelial, corneal or conjunctival edema. Further, the presence of disclosed ophthalmic solutions upon the cornea scavenges intraoperative free radicals, enhances postoperative comfort, increases rapidity of visual recovery, reduces postoperative corneal cell damage and staining, improves postoperative comfort, and enhances the quality of the patient's tear film.

Disclosed ophthalmic compositions are used as a deturgescent to reduce edema, promote cell membrane stabilization and protection, and scavenge free radicals. The key agent in the ophthalmic compositions of this invention is chondroitin sulfate which is hereby recognized as indicated as a deturgescent which functions to reduce edema, promote cell membrane stabilization and protection, and scavenge free radicals.

Still other embodiments of the disclosure are within the scope of the following claims.

Commercial Products/Kits

The present compositions can be finished as a commercial product by the usual steps performed in the present field, for example by appropriate sterilization and packaging steps. For example, disclosed kits comprise a composition consisting of, consisting essentially of, or comprising:
a. a rho kinase inhibitor (RKI);
b. a lubricant or cell culture medium;
c. a deturgescent agent;
d. a glycosaminoglycan; and
e. a liquid carrier.

Further disclosed kits can comprise a composition consisting of, consisting essentially of, or comprising:
a. An RKI;
b. a buffered corneal preservation medium;
c. recombinant human insulin (cell metabolism enhancer);
d. glutathione (antioxidant, free-radical scavenger, enzyme cofactor);
e. stabilized L-glutamine;
f. ATP precursors;
g. nutrient cell supplements;
h. amino acids;
i. vitamins;
j. trace elements;
k. gentamicin;
l. streptomycin; and
m. phenol red (pH indicator).

Further disclosed kits can comprise a composition consisting of, consisting essentially of, or comprising:
a. an RKI;
b. a cell culture media such as Minimum Essential Medium (MEM);
c. a deturgescent agent such as dextran;
d. a glycosaminoglycan such as chondroitin sulfate;
e. a liquid carrier such as water;
f. a buffered corneal preservation medium;
g. recombinant human insulin (cell metabolism enhancer);
h. glutathione (antioxidant, free-radical scavenger, enzyme cofactor);
i. stabilized L-glutamine;
j. ATP precursors;
k. nutrient cell supplements;
l. amino acids;
m. vitamins;
n. trace elements;
o. gentamicin;
p. streptomycin; and
q. phenol red (pH indicator).

The compositions according to the present disclosure can be finally sterile-wrapped so as to retain sterility until use and packaged (e.g. by the addition of specific product information leaflets) into suitable containers (boxes, etc.).

According to further embodiments, the disclosed compositions can also be provided in kit form combined with other components necessary for the desired use. In embodiments, disclosed compositions can be provided in 30 mL PETG bottles/HDPE caps packaged in units of, for example, 6, 12, or 24 bottles.

Storage Systems

Disclosed embodiments comprise corneal storage, viewing and transport systems comprising disclosed compositions.

### EXAMPLES

The following non-limiting Examples are provided for illustrative purposes only in order to facilitate a more complete understanding of representative embodiments. This example should not be construed to limit any of the embodiments described in the present Specification.

### Example 1

### Production of Ophthalmic Solutions

Any effective method may be used to produce the ophthalmic compositions described herein. An example of a method for making an ophthalmic composition using a borate buffer follows:
a. Add to the manufacturing vessel 80% of the batch quantity of Purified Water.
b. Heat to 85-90° C.
c. While mixing, add the batch quantity of Dextran-40K. Make sure powder is not splashed on vessel wall above the water level.
d. Mix until all Dextran is dissolved and a clear solution is observed.
e. Discontinue heating and allow solution to start cooling.
f. While cooling, increase speed of mixer and add slowly and to vortex the batch quantity of Chondroitin Sulfate Sodium. Avoid formation of undissolved lumps. Mix until all is dissolved.
g. Add batch quantity of Glycerol. Rinse container with few milliliters of Purified Water and add to the manufacturing vessel to assure complete transfer of glycerol.
h. Add batch quantity of RKI.
i. Add batch quantity of Boric Acid. Mix to dissolve.
j. Add batch quantity of Sodium Borate (decahydrate). Mix to dissolve.
k. Qs with Purified Water. Mix to homogeneity.
l. Using appropriate filters (e.g. 5 to 25 micron filters) filter the solution to remove any undissolved particles.
m. Sterile filter through 0.20 micron filters.

### Example 2

### Composition Use in a Corneal Transplant

Prior to transplantation, a donor cornea is “soaked” in a disclosed composition for 12-24 hours. This “pre-loads” the tissue with an RKI to accelerate healing post-transplant and increase endothelia cell count.

### Example 3

### Composition Use in Rehabilitating the Ocular Surface

A disclosed composition is used for rehabilitating the ocular surface before contact lens wear. Prior to applying the contact lens, a disclosed composition is applied to the cornea as an eye drop.

### Example 4

### Composition Use in Rehabilitating the Ocular Surface

A disclosed composition is used for rehabilitating the ocular surface after contact lens wear. After removing the contact lens, a disclosed composition is applied to the cornea as an eye drop.

### Example 5

### Composition Use in Enhancing Contact Lens Comfort

A disclosed composition is used for enhancing contact lens comfort. Prior to applying the contact lens, a disclosed composition is applied to the cornea as an eye drop.

### Example 6

### A Randomized, Open-Label, Parallel-Group Study of Two Dosing Regimens of Netarsudil in Patients with Fuchs Corneal Dystrophy

Fuchs Corneal Dystrophy (FCD) is a slowly progressive corneal disease, and is the most common indication for corneal transplants worldwide. A typical patient presents with blurred vision, glare, and a hazy cornea from corneal edema. The only definitive treatment option to restore vision is surgery. There is a large unmet need for pharmacologic alternatives to surgery

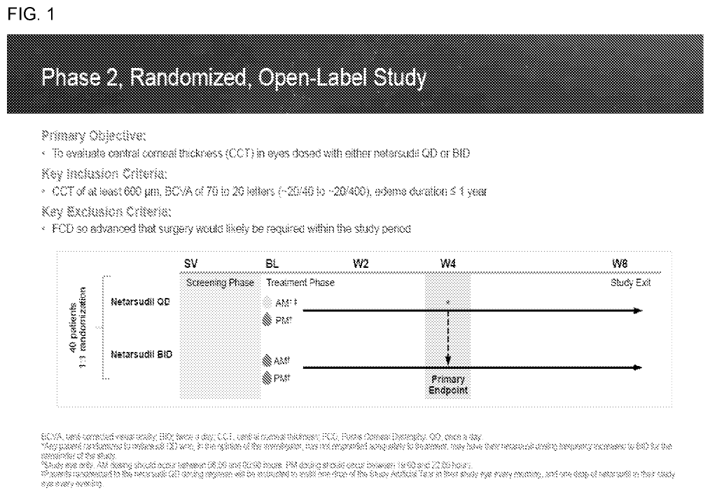

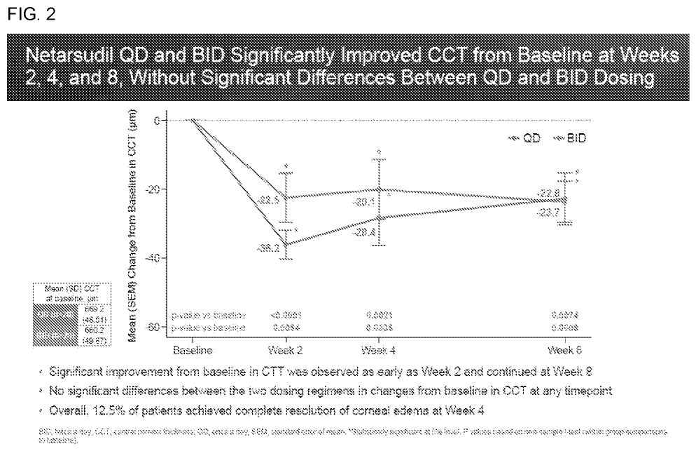

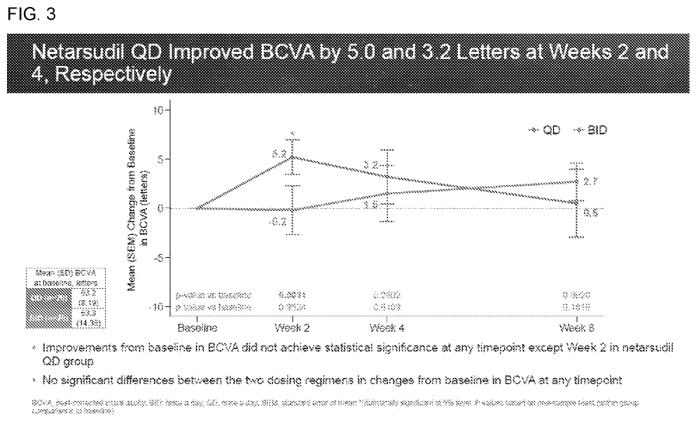

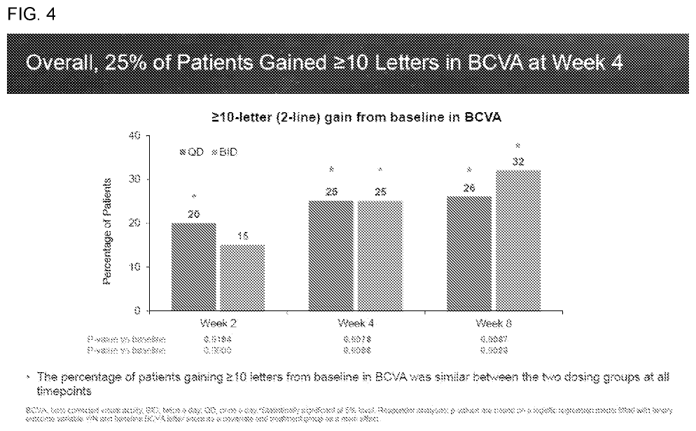

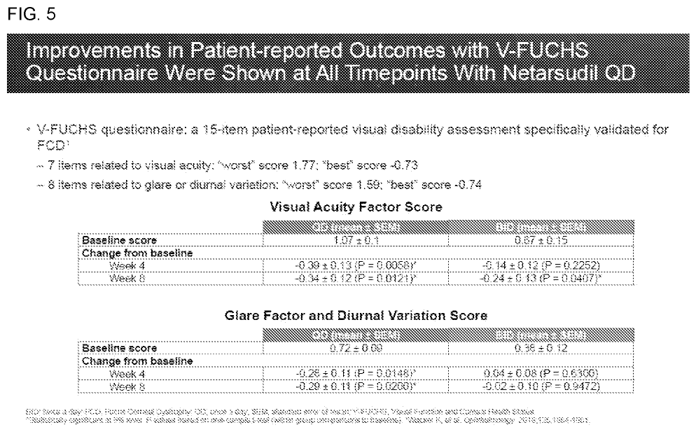

Purpose of this study: evaluate the therapeutic potential of two dosing regimens of netarsudil, a Rho kinase inhibitor, to reduce corneal edema and improve visual acuity in patients with FCD. The study is graphically shown in FIG. 1.
a. Netarsudil significantly reduced corneal edema and improved vision in patients with FCD. QD dosing was as effective as BID dosing (FIG. 2).
b. Significant improvements in corneal thickness at all timepoints for QD or BID dosing (FIG. 3).
c. Complete resolution of edema in 12.5% of patients at Week 4 (BID+QD) (FIG. 2).
d. 25% of patients gained letters from baseline in BCVA at Week 4 (BID+QD) (FIG. 4).
e. Improvements in corneal edema and vision observed as early as Week 2 and continued at Week 8 (FIG. 4).
f. Improvements in validated patient reported outcome questionnaire shown at all timepoints with netarsudil QD dosing (FIG. 5).
g. Netarsudil was well tolerated at both dosing regimens.
h. Most common ocular TEAE was hyperemia accounting for 3/9 and 6/17 total ocular TEAE in the QD and BID groups, respectively.
i. Reticular edema developed in 1 patient in BID group, which resolved with treatment discontinuation.

Further studies are warranted to explore the therapeutic potential of netarsudil to delay or prevent the need for surgery in patients with corneal edema due to FCD

In closing, it is to be understood that although aspects of the present Specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to, or alternative configurations of, the disclosed subject matter can be made in accordance with the teachings herein without departing from the spirit of the present Specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the Specification is deemed to contain the group as modified thus fulfilling the written description of all Markush groups used in the appended claims.

Unless otherwise indicated, all numbers expressing a characteristic, item, quantity, parameter, property, term, and so forth used in the present Specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present Specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. An ophthalmic composition comprising:
a) a Rho kinase inhibitor (RKI) in an amount of 1 mM to 100 mM;
b) glycerol in a concentration of from 0.1% to 5.0%;
c) dextran in a concentration of from 1.0% to 10.0%;
d) chondroitin sulfate in a concentration of from 0.1% to 5.0%; and
e) water or a cell culture medium.

2. The ophthalmic composition of claim 1, wherein the RKI is present in an amount of 4 mM to 16 mM, glycerol is present in a concentration of from 0.3% to 1.7%, dextran is present in a concentration of from 3.0% to 7.0%, and chondroitin sulfate is present in a concentration of from 0.5% to 4.5%.

3. The ophthalmic composition of claim 1, wherein the RKI is present in an amount of 8 mM to 12 mM, glycerol is present in a concentration of from 0.6% to 1.4%, dextran is present in a concentration of from 4.0% to 6.0%, and chondroitin sulfate is present in a concentration of from 1.5% to 3.5%.

4. The ophthalmic composition of claim 1, wherein the RKI is present in an amount of 9 mM to 11 mM, glycerol is present in a concentration of from 0.8% to 1.2%, dextran is present in a concentration of from 4.5% to 5.5%, and chondroitin sulfate is present in a concentration of from 2.0% to 3.0%.

5. The ophthalmic composition of claim 4, wherein chondroitin sulfate is present in a concentration of from 2.3% to 2.7%.

6. The ophthalmic composition of claim 1, wherein glycerol is present in a concentration of 1.0%, dextran is present in a concentration of 5.0%, and chondroitin sulfate is present in a concentration of 2.5%.

7. An ophthalmic composition consisting essentially of an RKI in an amount of 1 mM to 100 mM, glycerol in a concentration of from 0.1% to 5.0%; dextran in a concentration of from 1.0% to 10.0%; chondroitin sulfate in a concentration of from 0.1% to 5.0%; water; a buffer; and pH 6 to 8.

8. The ophthalmic composition of claim 7, wherein the pH of the composition is from 7.0 to 7.4.

9. The ophthalmic composition of claim 7, wherein the RKI is present in an amount of 5 mM to 20 mM, glycerol is present in a concentration of from 0.3% to 1.7%, dextran is present in a concentration of from 3.0% to 7.0%, and chondroitin sulfate is present in a concentration of from 0.5% to 4.5%.

10. The ophthalmic composition of claim 7, wherein the RKI is present in an amount of 8 mM to 12 mM, glycerol is present in a concentration of from 0.6% to 1.4%, dextran is present in a concentration of from 4.0% to 6.0%, and chondroitin sulfate is present in a concentration of from 1.5% to 3.5%.

11. The ophthalmic composition of claim 7, wherein the RKI is present in an amount of 9 mM to 11 mM, glycerol is present in a concentration of from 0.8% to 1.2%, dextran is present in a concentration of from 4.5% to 5.5%, and chondroitin sulfate is present in a concentration of from 2.0% to 3.0%.

12. The ophthalmic composition of claim 11, wherein chondroitin sulfate is present in a concentration of from 2.3% to 2.7%.

13. The ophthalmic composition of claim 7, wherein the RKI is present in an amount of 10 mM, glycerol is present in a concentration of 1.0%, dextran is present in a concentration of 5.0%, and chondroitin sulfate is present in a concentration of 2.5%.

14. The ophthalmic composition of claim 7, wherein the buffer comprises sodium borate and boric acid.

15. An ophthalmic composition consisting of an RKI in an amount of from 1 to 100 mM; glycerol in a concentration of from 0.1% to 5.0%; dextran in a concentration of from 1.0% to 10.0%; chondroitin sulfate in a concentration of from 0.1% to 5.0%; and water.

16. The ophthalmic composition of claim 15, wherein the RKI is present in an amount of from 2 mM to 20 mM, glycerol is present in a concentration of from 0.3% to 1.7%, dextran is present in a concentration of from 3.0% to 7.0%, and chondroitin sulfate is present in a concentration of from 0.5% to 4.5%.

17. The ophthalmic composition of claim 15, wherein the RKI is present in an amount of from 5 mM to 15 mM, glycerol is present in a concentration of from 0.6% to 1.4%, dextran is present in a concentration of from 4.0% to 6.0%, and chondroitin sulfate is present in a concentration of from 1.5% to 3.5%.

18. The ophthalmic composition of claim 15, wherein the RKI is present in an amount of from 8 mM to 12 mM, glycerol is present in a concentration of from 0.8% to 1.2%, dextran is present in a concentration of from 4.5% to 5.5%, and chondroitin sulfate is present in a concentration of from 2.0% to 3.0%.

19. The ophthalmic composition of claim 18, wherein chondroitin sulfate is present in a concentration of from 2.3% to 2.7%.

20. The ophthalmic composition of claim 15, wherein the RKI is present in an amount of 10 mM, glycerol is present in a concentration of 1.0%, dextran is present in a concentration of 5.0%, and chondroitin sulfate is present in a concentration of 2.5%.

21. A method of performing ophthalmic surgery comprising the steps of:
a. pretreating the cornea of a patient's eye immediately before performing ophthalmic surgery by administering an ophthalmic composition comprising;
(1) an RKI in an amount of from 1 mM to 100 mM;
(2) glycerol in a concentration of from 0.1% to 5.0%;
(3) dextran in a concentration of from 1.0% to 10.0%;
(4) chondroitin sulfate in a concentration of from 0.1% to 5.0%; and,
(5) water; and
b. performing ophthalmic surgery on the patient's eye through the film of the ophthalmic composition upon the cornea.

22. The method of claim 21, wherein, in the ophthalmic composition that is administered, the RKI is present in an amount of from 2 mM to 20 mM, glycerol is present in a concentration of from 0.3% to 1.7%, dextran is present in a concentration of from 3.0% to 7.0%, and chondroitin sulfate is present in a concentration of from 0.5% to 4.5%.

23. The method of claim 21, wherein, in the ophthalmic composition that is administered, the RKI is present in an amount of from 5 mM to 15 mM, glycerol is present in a concentration of from 0.6% to 1.4%, dextran is present in a concentration of from 4.0% to 6.0%, and chondroitin sulfate is present in a concentration of from 1.5% to 3.5%.

24. The method of claim 21, wherein, in the ophthalmic composition that is administered, the RKI is present in an amount of from 8 mM to 12 mM, glycerol is present in a concentration of from 0.8% to 1.2%, dextran is present in a concentration of from 4.5% to 5.5%, and chondroitin sulfate is present in a concentration of from 2.0% to 3.0%.

25. The method of claim 21, wherein, in the ophthalmic composition that is administered, chondroitin sulfate is present in a concentration of from 2.3% to 2.7%.

26. The method of claim 21, wherein, in the ophthalmic composition that is administered, the RKI is present in an amount of 10 mM, glycerol is present in a concentration of 1.0%, dextran is present in a concentration of 5.0%, and chondroitin sulfate is present in a concentration of 2.5%.

27. The method of claim 21, wherein the ophthalmic composition further comprises a buffer and a tonicity modulating agent.

28. A method of promoting healing of ocular tissue of a mammal after trauma to such ocular tissue, the method comprising administering to the ocular tissue before, during, or after the trauma an effective amount of an ophthalmic composition comprising: an RKI in an amount of from 1 mM to 100 mM, glycerol in a concentration of from 0.1% to 5.0%; dextran in a concentration of from 1.0% to 10.0%; chondroitin sulfate in a concentration of from 0.1% to 5.0%; and water.

29. The method of claim 28, wherein, in the ophthalmic composition that is administered, the RKI is present in an amount of from 2 mM to 20 mM, glycerol is present in a concentration of from 0.3% to 1.7%, dextran is present in a concentration of from 3.0% to 7.0%, and chondroitin sulfate is present in a concentration of from 0.5% to 4.5%.

30. The method of claim 28, wherein, in the ophthalmic composition that is administered, the RKI is present in an amount of from 5 mM to 15 mM, glycerol is present in a concentration of from 0.6% to 1.4%, dextran is present in a concentration of from 4.0% to 6.0%, and chondroitin sulfate is present in a concentration of from 1.5% to 3.5%.

31. The method of claim 28, wherein, in the ophthalmic composition that is administered, the RKI is present in an amount of from 8 mM to 12 mM, glycerol is present in a concentration of from 0.8% to 1.2%, dextran is present in a concentration of from 4.5% to 5.5%, and chondroitin sulfate is present in a concentration of from 2.0% to 3.0%.

32. The method of claim 28, wherein, in the ophthalmic composition that is administered, chondroitin sulfate is present in a concentration of from 2.3% to 2.7%.

33. The method of claim 28, wherein, in the ophthalmic composition that is administered, the RKI is present in an amount of 10 mM, glycerol is present in a concentration of 1.0%, dextran is present in a concentration of 5.0%, and chondroitin sulfate is present in a concentration of 2.5%.

34. The method of claim 28, wherein the ophthalmic composition further comprises a buffer and a tonicity modulating agent.

35. A method of fully or partially rehabilitating stressed or damaged ocular tissue to a normal state, the method comprising administering to the stressed or damaged ocular tissue an effective amount of an ophthalmic composition comprising: an RKI in an amount of from 1 mM to 100 mM; glycerol in a concentration of from 0.1% to 5.0%; dextran in a concentration of from 1.0% to 10.0%; chondroitin sulfate in a concentration of from 0.1% to 5.0%; and water.

36. The method of claim 35, wherein, in the ophthalmic composition that is administered, the RKI is present in an amount of from 2 mM to 20 mM, glycerol is present in a concentration of from 0.3% to 1.7%, dextran is present in a concentration of from 3.0% to 7.0%, and chondroitin sulfate is present in a concentration of from 0.5% to 4.5%.

37. The method of claim 35, wherein, in the ophthalmic composition that is administered, the RKI is present in an amount of from 5 mM to 15 mM, glycerol is present in a concentration of from 0.6% to 1.4%, dextran is present in a concentration of from 4.0% to 6.0%, and chondroitin sulfate is present in a concentration of from 1.5% to 3.5%.

38. The method of claim 35, wherein, in the ophthalmic composition that is administered, the RKI is present in an amount of from 8 mM to 12 mM, glycerol is present in a concentration of from 0.8% to 1.2%, dextran is present in a concentration of from 4.5% to 5.5%, and chondroitin sulfate is present in a concentration of from 2.0% to 3.0%.

39. The method of claim 35, wherein, in the ophthalmic composition that is administered, chondroitin sulfate is present in a concentration of from 2.3% to 2.7%.

40. The method of claim 35, wherein, in the ophthalmic composition that is administered, the RKI is present in an amount of 10 mM, glycerol is present in a concentration of 1.0%, dextran is present in a concentration of 5.0%, and chondroitin sulfate is present in a concentration of 2.5%.

41. The method of claim 35, wherein the ophthalmic composition further comprises a buffer and a tonicity modulating agent.

42. A method of preserving tissue, the method comprising the steps of: providing excised mammalian tissue to be preserved; providing a liquid composition comprising an RKI in an amount of from 1 mM to 100 mM, glycerol in a concentration of from 0.1% to 5.0%, dextran in a concentration of from 1.0% to 10.0%, chondroitin sulfate in a concentration of from 0.1% to 5.0%, and water; and placing the excised mammalian tissue to be preserved into the liquid composition under in vitro conditions.

43. The method of claim 42, wherein the excised mammalian tissue to be preserved is ophthalmic tissue.

44. The method of claim 43, wherein ophthalmic tissue to be preserved is corneal tissue.

45. The method of claim 44, wherein corneal tissue to be preserved is a cornea.

46. The method of claim 42, wherein the liquid composition and tissue to be preserved are stored at a temperature of about 0° C.-40° C.

47. The method of claim 42, further comprising the step of replenishing the liquid composition in which the tissue to be preserved is placed.

48. The method of claim 47, wherein the step of replenishing is a batch replenishment.

49. The method of claim 47, wherein the step of replenishing is a continuous replenishment.

50. The method of claim 45, wherein the cornea is placed in a volume of from about 50 ml to about 500 ml of the liquid composition.

51. The method of claim 42, further comprising the step of subsequently implanting the excised mammalian tissue preserved in the liquid composition.

52. The method of claim 51, wherein the tissue is a cornea.

53. The method of claim 42, wherein the liquid composition is an ophthalmic composition consisting essentially of an RKI in an amount of from 2 mM to 20 mM, glycerol in a concentration of from 0.1% to 5.0%, dextran in a concentration of from 1.0% to 10.0%, chondroitin sulfate in a concentration of from 0.1% to 5.0%, and water.

54. The method of claim 53, wherein the liquid composition is an ophthalmic composition and the RKI is present in an amount of from 5 mM to 15 mM, glycerol is present in a concentration of from 0.3% to 1.7%, dextran is present in a concentration of from 3.0% to 7.0%, and chondroitin sulfate is present in a concentration of from 0.5% to 4.5%.

55. The method of claim 53, wherein the liquid composition is an ophthalmic composition and the RKI is present in an amount of from 7 mM to 13 mM, glycerol is present in a concentration of from 0.6% to 1.4%, dextran is present in a concentration of from 4.0% to 6.0%, and chondroitin sulfate is present in a concentration of from 1.5% to 3.5%.

56. The method of claim 53, wherein the liquid composition is an ophthalmic composition and the RKI is present in an amount of from 8 mM to 12 mM, glycerol is present in a concentration of from 0.8% to 1.2%, dextran is present in a concentration of from 4.5% to 5.5%, and chondroitin sulfate is present in a concentration of from 2.0% to 3.0%.

57. The method of claim 56, wherein the liquid composition is an ophthalmic composition and chondroitin sulfate is present in a concentration of from 2.3% to 2.7%.

58. The method of claim 53, wherein the liquid composition is an ophthalmic composition and the RKI is present in an amount of 10 mM, glycerol is present in a concentration of 1.0%, dextran is present in a concentration of 5.0%, and chondroitin sulfate is present in a concentration of 2.5%.

59. The method of claim 42, wherein the liquid composition is an ophthalmic composition consisting essentially of: an RKI in an amount of from 2 mM to 20 mM, glycerol in a concentration of from 0.1% to 5.0%, dextran in a concentration of from 1.0% to 10.0%, chondroitin sulfate in a concentration of from 0.1% to 5.0%, water, a buffer, and pH 6 to 8.

60. The method of claim 59, wherein the liquid composition is an ophthalmic composition and the pH of the composition is from 7.0 to 7.4.

61. The method of claim 59, wherein the liquid composition is an ophthalmic composition and the RKI is present in an amount of from 5 mM to 15 mM, glycerol is present in a concentration of from 0.3% to 1.7%, dextran is present in a concentration of from 3.0% to 7.0%, and chondroitin sulfate is present in a concentration of from 0.5% to 4.5%.

62. The method of claim 59, wherein the liquid composition is an ophthalmic composition and the RKI is present in an amount of from 8 mM to 12 mM, glycerol is present in a concentration of from 0.6% to 1:4%, dextran is present in a concentration of from 4.0% to 6.0%, and chondroitin sulfate is present in a concentration of from 1.5% to 3.5%.

63. The method of claim 59, wherein the liquid composition is an ophthalmic composition and the RKI is present in an amount of from 9 mM to 11 mM, glycerol is present in a concentration of from 0.8% to 1.2%, dextran is present in a concentration of from 4.5% to 5.5%, and chondroitin sulfate is present in a concentration of from 2.0% to 3.0%.

64. The method of claim 63, wherein the liquid composition is an ophthalmic composition and chondroitin sulfate is present in a concentration of from 2.3% to 2.7%.

65. The method of claim 58, wherein the liquid composition is an ophthalmic composition and the buffer is sodium borate and boric acid.

66. The method of claim 42, wherein the liquid composition further includes from about 0.01% to about 20% of mannitol.

67. The method of claim 42, wherein the liquid composition further includes about 0.5% of mannitol.

68. The method of claim 42, wherein the excised mammalian tissue is ophthalmic tissue which is excised from a mammalian donor, preserved in vitro, and subsequently implanted into a recipient.

69. A method of improving the performance of an ophthalmic solution by augmenting the solution with between 1 mM and 100 mM of an RKI.
